(19) United States
(12) Patent Application Publication
(10) Pub. No.: US 2019/0246997 A1
(43) Pub. Date: Aug. 15, 2019

(54) CONSOLE AND X-RAY IMAGING APPARATUS HAVING THE SAME

(71) Applicant: SHIMADZU CORPORATION, KYOTO-SHI (JP)

(72) Inventor: Masahiro Tanaka, KYOTO-SHI (JP)

(73) Assignee: SHIMADZU CORPORATION, KYOTO-SHI (JP)

(21) Appl. No.: 15/896,539

(22) Filed: Feb. 14, 2018

(51) Int. Cl. A61B 6/00; H05G 1/28

(52) U.S. Cl. CPC A61B 6/46; H05G 1/28

(57) ABSTRACT

A console for an ex-ray imaging apparatus improves operability and includes in combination an operation panel 26 and a recording button 26b on an imaging system operation lever 26a. In use, the operator grips the imaging system operation lever 26a while video imaging allowing the operator to operate a recording button 26b without unlinking a hand from the imaging system operation lever 26a while watching the display element 25 without changing the line of sight to the operation panel 26. Accordingly, the operator can start recording immediately without changing the line of the own sight, so that the scene for diagnosis is accurately recorded.

### CROSS REFERENCE TO RELATED APPLICATIONS

This application relates to, and but does not claim priority from, Ser. No.: JP 2015-158952 which was filed Aug. 11, 2015 and published on Feb. 16, 2017 as JP P2017-035325, the entire contents of which are incorporated herein by reference.

### FIGURE SELECTED FOR PUBLICATION

FIG. 5.

### BACKGROUND OF THE INVENTION

### Field of the Invention

The present invention relates to a console that is applied to a video imaging and an X-ray imaging apparatus having the same.

### Background

FIG. 9 is a schematic diagram illustrating a conventional radiation imaging apparatus. Referring to FIG. 9, such apparatus comprises a table 52 on which a subject is lorded, a radiation source 53 that irradiates a radiation, and a detector 54 that detects the radiation. Such radiation imaging apparatus comprises an imaging mode that is ready to take a real-time fluoroscopy of a subject and creates a video of fluoroscopic images so that the present inside structure of the subject can be provided (e.g., refer to the Patent Document 1). Such imaging is called a fluoroscopy imaging or a DA imaging, in which radiation images are taken as a video. Such video imaging is carried out in a variety of medical aspects. An example of performing such video imaging is a fluoroscopic swallowing exam. The fluoroscopic swallowing exam is an exam for the purpose of studying whether a subject can swallow food normally, in which the video is taken while the subject is swallowing a pseudo-food containing barium.

Referring to FIG. 10, a console that is installed to the apparatus is shown. The conventional console comprises an operation lever (stick) for an imaging system (imaging system operation lever) and a recording button. The operation lever is a human interface that is used to input the operation of the operator, and when such lever is operative, the imaging system that comprises a radiation source 53 and a detector 54 shifts relative to a table 52. The imaging system is shiftable even while taking a video.

The recording button is also a human interface that is used to input the operation of the operator, and when such button is operative, the recording of the video starts. The video is recorded as a video file thereof, and is applied for diagnosis following the imaging as the result of the fluoroscopic swallowing exam. When the system records automatically the video imaging from the begging to the end, the preparation work for the imaging, which is unrelated to the diagnosis, also is recorded as the video file. Accordingly, relative to the conventional apparatus, the operator can direct to start recording the video while imaging the video. Such recording button is the button that is operative while imaging the video.

### RELATED PRIOR ART DOCUMENTS

### Patent Document

Patent Document 1: JP Patent Published 2007-185238

### ASPECTS AND SUMMARY OF THE INVENTION

### Objects to be Solved

However, the following problems are remained to be solved in the conventional system.

Specifically, it is deemed that the operability of the console according to the aspect of the conventional structure is not good at the beginning of recording the video.

The operator involved in such video imaging watches the monitor displaying the video. The reason is that the operator must watch the ever-changing fluoroscopic image of the subject. In addition, the operator involved in the operation is gripping the imaging system operation lever by the operator's own hand. When tracking (monitoring) the movement of the pseudo-food containing barium that the subject swallows, the imaging system must shift along with the subject.

Referring to FIG. 11, the operator acts according to the flow chart when the operator realizes that the recording must starts in the middle of video imaging. Referring to FIG. 11, the operator is inefficient relative to the operation. When the operator realizes that the video recording is needed, first, the operator must shift the line of the own sight from the monitor to the console. At this time, the operator must be aware of the location of the recording button on the console. Then the operator unlinks a hand from the imaging system operation lever and moves the hand to the recording button. At last, the operator presses down the button.

While the operator is inefficient relative to the operation, the time passes by. Accordingly, with regard to the console having the conventional structure, the operator is unable to start the video recording at once, so that the operator cannot record satisfactorily the scene needed for diagnosis using a video.

Considering such circumstances, the purpose of the present invention is to provide a console having a good operability, and by which the video recording can start at once.

### Means for Solving the Problem

The present invention comprises the following structures to solve the above problem.

Specifically, a console, according to the aspect of the present invention, is the console that a radiation imaging apparatus that comprises; an imaging system that consists of a radiation source that irradiate a radiation and a detector that detects the radiation that transmits a subject, and a display means that displays fluoroscopic images of the subject as a real-time video; comprises an imaging system operation lever that an operator inputs a directive relative to a shift of the imaging system along with the subject, and a storing directive input means that the operator inputs the directive to store a video, wherein the storing directive input means is installed to the imaging system operation lever. As used herein, a video includes at least one of a still image and a series of still images.

### Action and Effect

The console according to the aspect of the present invention comprises the imaging system operation lever having the storing directive input means. The operator grips the imaging system operation lever by a hand while the video imaging, so that the operator can operate the storing directive input means without unlinking the hand from the imaging system operation lever. The operator is, in advance, aware of that the storing directive input means is installed to the imaging system operation lever, so that the operator can operate the storing directive input means while watching the display means without changing the line of sight to the console. Accordingly, with regard to the console according to the aspect of the present invention, when the operator starts recording the video, the operator can start recording immediately without changing the line of the own sight, so that the scene needed for the diagnosis using the video can be absolutely recorded.

Further, according to the above console, it is further preferable that the storing directive input means is installed to a superior portion (top portion) of the imaging system operation lever.

### Action and Effect

The above aspect illustrates further specifically the console of the present invention. Given the storing directive input means is installed to the superior portion of the imaging system operation lever, it can be prevented that the operator erroneously operates the storing directive input means while operating the imaging system operation lever.

In addition, the above console may comprise a collimator operation input means that the operator inputs the directive relative to an aperture of the collimator that limits the broadening of the radiation that the radiation source irradiates.

In addition, the above console may comprise a table operation input means that the operator inputs the directive relative to a tilt of the table that the subject is loaded.

### Action and Effect

The above aspect illustrates further specifically the console of the present invention. The console of the present invention may comprise the other human interface than the imaging system operation lever.

In addition, the radiation imaging apparatus, comprising the console according to the aspect of the present invention, may store the video from the point when the operator inputs the directive through the storing directive input means, or may store the video until the point when the operator inputs the directive through the storing directive input mean. The aspect of the present invention is variable in accordance with necessity.

### Effect of the Invention

The console according to the aspect of the present invention comprises the imaging system operation lever having the storing directive input means. The operator grips the imaging system operation lever by a hand while the video imaging, so that the operator can operate the storing directive input means without unlinking the hand from the imaging system operation lever. The operator is, in advance, aware of that the storing directive input means is installed to the imaging system operation lever, so that the operator can operate the storing directive input means while watching the display means without changing the line of sight to the console. Accordingly, with regard to the console according to the aspect of the present invention, when the operator starts the video recording, the operator can start recording immediately without changing the line of own sight, so that the scene needed for the diagnosis using the video can be absolutely recorded.

### BRIEF DESCRIPTION OF THE DRAWINGS

FIG. 1 is a functional block diagram illustrating the entire structure of an X-ray imaging apparatus according to the Embodiment 1.

FIG. 2 is a schematic diagram illustrating a shift of an imaging system according to the aspect of the Embodiment 1.

FIG. 3 is a schematic diagram illustrating a rotation of a table according to the aspect of Embodiment 1.

FIG. 4 is a schematic diagram illustrating the opening-and-closing of a collimator according to the aspect of the Embodiment 1.

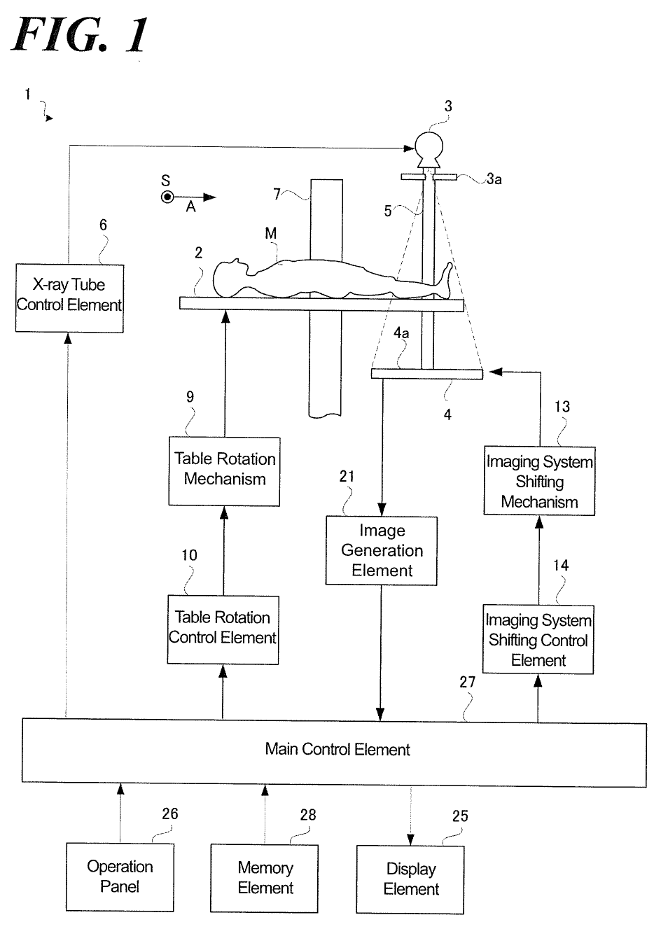

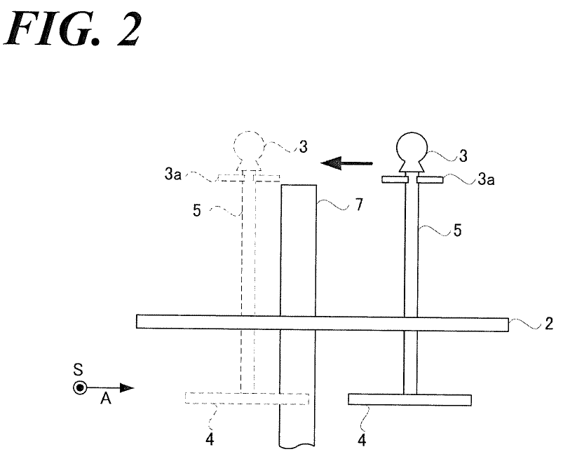

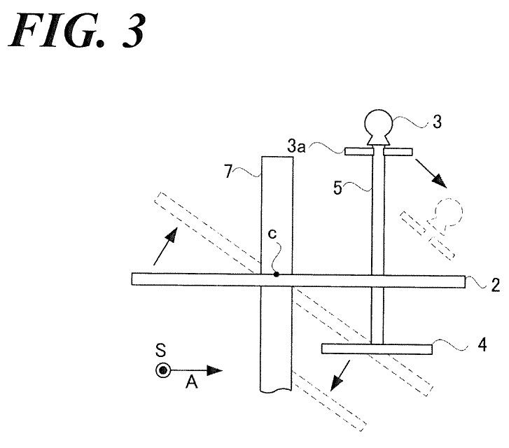

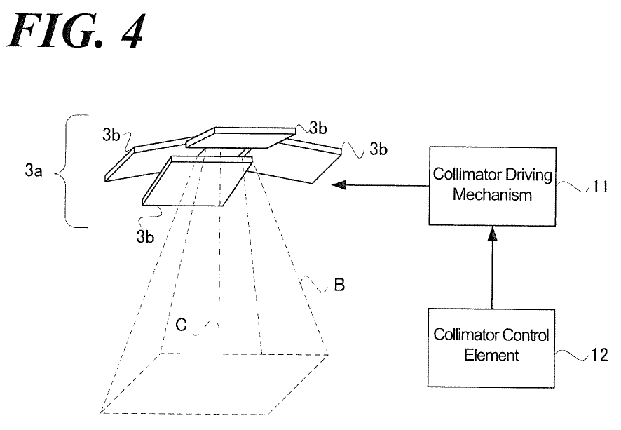

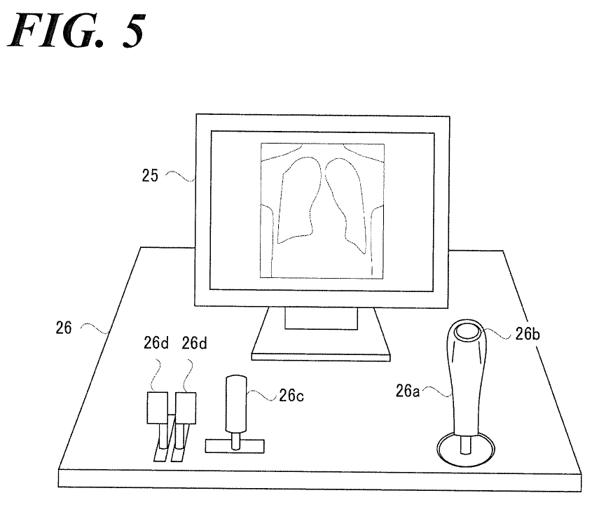

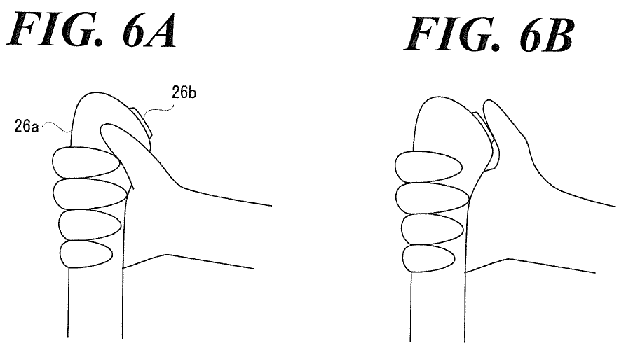

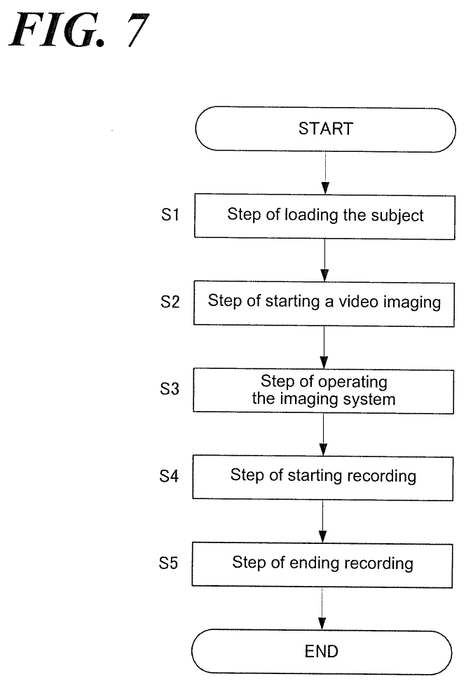

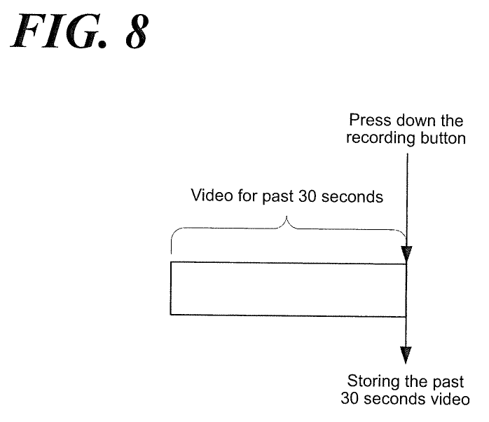

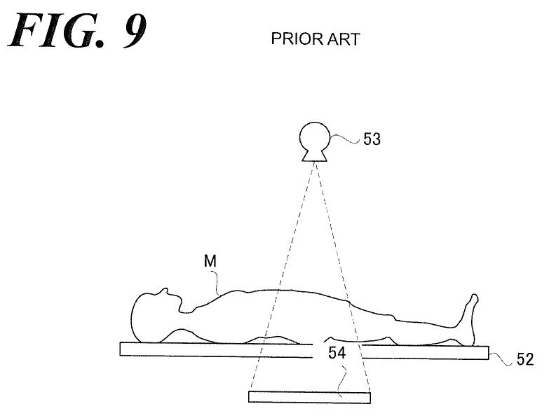

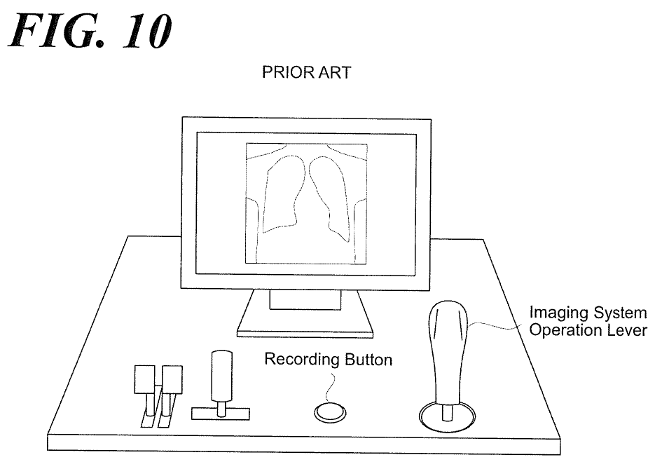

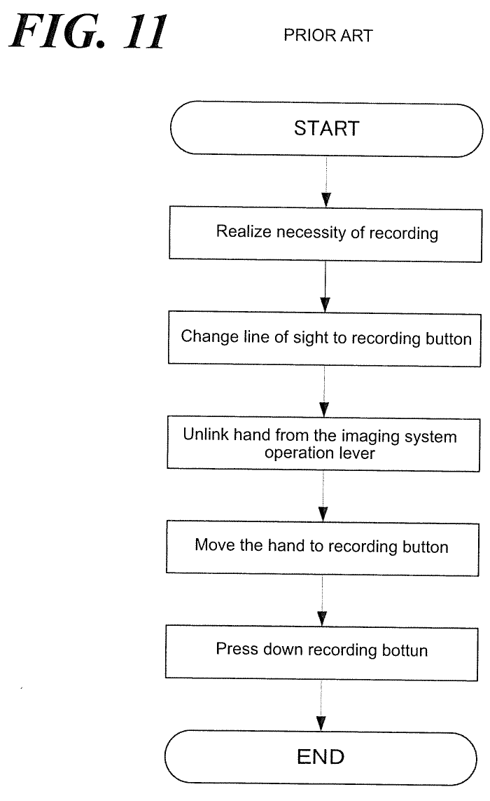

FIG. 5 is a schematic diagram illustrating a console according to the aspect of Embodiment 1.

FIGS. 6A, 6B are schematic views illustrating a characteristic of the console according to the aspect of Embodiment 1.

FIG. 7 is a flow-chart illustrating an exam using the apparatus according to the aspect of Embodiment 1.

FIG. 8 is a timing chart illustrating a structure according to the aspect of the alternative Embodiment 1 of the present invention.

FIG. 9 is a schematic diagram illustrating a conventional radiation imaging apparatus.

FIG. 10 is a schematic diagram illustrating a conventional console.

FIG. 11 is a flow-chart illustrating the exam using the conventional apparatus.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements, modules or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

It will be further understood by those of skill in the art that the apparatus and devices and the elements herein, without limitation, and including the sub components such as operational structures, circuits, communication pathways, and related elements, control elements of all kinds, display circuits and display systems and elements, any necessary driving elements, inputs, sensors, detectors, memory elements, processors, resistors, capacitors, switches, and any other electronic-circuit-related elements, and any combinations of these structures etc. as will be understood by those of skill in the art as also being identified as or capable of operating the systems and devices and subcomponents noted herein and structures that accomplish the functions without restrictive language or label requirements since those of skill in the art are well versed in related devices, computer and operational controls and technologies of radiographic devices and all their sub components, elements, modules, and programs, including various circuits, elements, and modules, and combinations thereof without departing from the scope and spirit of the present invention.

Although only a few embodiments have been disclosed in detail above, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art of x-ray imaging devices and the complex arrangements therein, including electronics engineers, software engineers, circuit design engineers and related individuals having advanced technical degrees, and as a result basic component elements will be easily understood by those of such skill in the art.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates a storage of or a transfer of a computer program from one place to another. A storage media may be any available media that can be accessed by a computer. By way of example, and not limitation, such computer-readable media can comprise RAM, ROM, EEPROM, CD-ROM or other optical disk storage, flash-memory, magnetic disk storage or other magnetic storage devices, or any other medium known to those of skill in the art of memory storage and x-ray apparatus design that can be used to carry or to store desired program code or image data or any other electronic memory media in the form of instructions or data structures and that can be accessed by a computer.

The memory storage can also be, alternatively, rotating magnetic hard disk drives, optical disk drives, or flash memory based storage drives or other such solid state, magnetic, or optical storage devices. Also, any connection is properly termed a computer-readable medium. For example, a disk if used herein includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of any computer-readable media of any form. The computer readable media can be an article comprising a machine-readable non-transitory or transitory (flash) tangible medium embodying result data or information indicative of instructions that when performed by one or more machines result in computer implemented operations comprising the actions described throughout this specification.

### Embodiment 1

Hereinafter, the inventor sets forth the Embodiment of the present invention. According to the aspect of the Embodiment, an X-ray corresponds to a radiation of the present invention. In addition, the FPD stands for Flat Panel Detector, and the video corresponds to the video of the present invention. Further, the inventor sets forth the following Embodiment relative to the fluoroscopic swallowing exam to enhance the characteristic of the console according to the aspect of the present Embodiment.

<Entire System of the X-Ray Imaging Apparatus>

First, the inventor sets forth the system of the X-ray imaging apparatus according to the aspect of the Embodiment 1. Referring to FIG. 1, the X-ray imaging apparatus 1 comprises a table 2 on which a subject M in the supine position is laid, an X-ray tube 3 to irradiate an X-ray is mounted above the table 2 and the FPD 4 to detect the X-ray that transmits through the subject M is mounted under the table 2. The FPD 4 has a rectangular shape with 4 sides along with either the axis direction A of the body or the side direction S of the body of the subject M. In addition, the X-ray tube 3 irradiates the X-ray quadrangular pyramid beam radiating out therefrom to the FPD 4. The X-ray tube 3 corresponds to the radiation source of the present invention.

A stand 5 supports the imaging system 3, 3a, 4 comprising the X-ray tube 3, the collimator 3a and the FPD 4. The stand 5 that is driven by the imaging system shifting mechanism 13 is shiftable in the body-axis direction A of the subject M relative to the table 2. Accordingly, the imaging system shifting mechanism 13 is the mechanism by which the X-ray tube 3 and the FPD4 move relative to the table 2 in an integrated manner in the longitudinal direction of the table 2. According to such shifting, the X-ray imaging position for the subject M can be changed. The imaging system shifting control element 14 is installed to control the imaging system shifting mechanism 13. FIG. 2 is illustrating the manner in which the imaging system shifting mechanism 13 shifts the imaging system 3,3a, 4 together with the stand 5. Accordingly, even when the imaging system shifting mechanism 13 shifts, the positional relationship between the X-ray tube 3, the collimator 3a, and the FPD 4 and the stand 5 does not change.

A table support member 7 is a member vertically extending from the floor surface of the examination room and supports the table 2 with freedom of the rotation thereof. Rotation of the table 2 can be brought into reality by a table rotation mechanism 9 installed to the table support 7. The table rotation control element 10 is installed to control the table rotation mechanism 9. The table rotation control element 10 controls the table rotation mechanism 9 in accordance with input through the operation panel 26.

FIG. 3 is illustrating the aspect in which the table 2 rotates in one direction around the axis C as the center thereof by the table rotation mechanism 9. The axis C is extending in the width direction (side direction S of body of the subject M) of the table 2. The table rotation mechanism 9 can also reversely rotate the table 2 that rotates in one direction. The imaging system 3, 3a, 4 rotates along with the table 2 while keeping the relative positional relationship with the table 2.

The collimator 3a that narrows (limits) the X-ray radiation range is installed the ray tube 3. The collimator 3a can adjust the aperture. Referring to FIG. 4, the collimator 3a comprises one pair of shielding-leaves (diaphragms) 3b that moves mirror-image-symmetrically on the basis of the axis C and another pair of shielding-leaves (diaphragms) 3b that also moves mirror-image-symmetrically on the basis of the axis C. The collimator 3a shifts the diaphragms 3b so that not only the cone-shape X-ray beam B can be irradiated to a whole plane of the detection plane 4a of the FPD 4, but also, for example, the fan-shape X-ray beam B can be irradiated only to the center portion of the detection plane 4a of the FPD 4. Further, the axis C is an axis specifying the center of the X-ray beam B. In addition, one of the two pairs of diaphragms 3b adjusts broadening of the quadrangular pyramid shape X-ray beam in the body-axis direction A, and the other one pair of the diaphragms 3b adjusts broadening of the X-ray beam in the side direction S of the body. When the X-ray tube 3 shifts, the collimator 3a also shifts along with the X-ray tube 3. One pair of the diaphragms 3b blocks the X-ray.

Referring to FIG. 4, the collimator driving mechanism 11 brings the opening-and-closing of such diaphragms 3b into reality. The collimator driving mechanism 11 comprise specifically such as a stepping motor and so forth. The collimator control element 12 is operative to control the collimator driving mechanism 11.

The purpose of an X-ray tube control element 6 is to control parameters including a tube current electricity of the X-ray tube 3, a tube electric voltage and an irradiation and exposure time. The FPD4 detects an X-ray that the X-ray 3 tube irradiates and transmits through the subject M, and generates the detection signal by detecting the X-ray. Such detection signals are output to the image generation element 21 that generates a video of the fluoroscopic images of the subject M.

The purpose of the display element 25 is to display each image acquired by the X-ray imaging. The purpose of the operation panel 26 is for the operator to input the directive of the operator relative to the aperture of the collimator 3a, the directive of the operator relative to the rotation of the table 2 and the directive of the operator relative to the shift of the imaging system 3, 3a, 4. In addition, the purpose of the main control element 27 is to control comprehensively each control element. The main control element 27 comprises a CPU, and brings each control element 6, 10, 12,14 and the image generation element 21 into reality by executing a variety of programs. In addition, the above each element can be executed separately by an arithmetic device to run each element. The memory element 28 stores all parameters needed for the imaging. The display element 25 displays the real-time video of the fluoroscopic images of the subject M and corresponds to the display means of the present invention, and the operation panel 26 corresponds to the console of the present invention.

FIG. 5 is a schematic view illustrating the operation panel 26 of the present invention. The operation panel 26 comprises the imaging system operation lever 26a through which the operator inputs the directive relative to the shift of the imaging system 3, 3a, 4. The operator can tilt the imaging system operation lever 26a. The tilting direction of the imaging system operation lever 26a and the tilting angle thereof imply the directive relative to the shifting direction of the imaging system 3, 3a, 4 and the shifting rate thereof. The imaging system shifting control element 14 controls the imaging system shifting mechanism 13 in accordance with the input by the operator through the imaging system operation lever 26a. The imaging system operation lever 26a of the present invention is used to input the directive of the operator relative to the shift of the imaging system 3, 3a, 4 for the subject.

The operation panel 26 comprises the table operation lever 26c through which the operator inputs the directive relative to the rotation of the table 2. The operator can tilt the table operation lever 26c. The tilting direction of the table operation lever 26c and the tilting angle thereof imply the directive relative to the rotation direction of the table 2 and the rotation rate thereof. The table rotation control element 10 controls the table rotation mechanism 9 in accordance with the input by the operator through the table operation lever 26c. The table operation lever 26c is used to input the directive of the operator relative to the tilt of the table 2 that the subject M is loaded, and corresponds to the table operation input means of the present invention.

And the operation panel 26 comprises the aperture change lever 26d through which the operator inputs the directive relative to the aperture change of the collimator 3a. The operator can slide the aperture change lever 26d. The position of the aperture change lever 26d implies the aperture of the collimator 3a. The collimator control element 12 controls the collimator driving mechanism 11 according to the input of the operator through the aperture change lever 26d. The collimator 3a comprises two pairs of diaphragms 3b. Accordingly, the operation panel 26 comprises the aperture change lever 26d that controls the opening-and-closing of one pair of the diaphragms 3b, and in addition, comprises another change lever 26d that controls the opening-and-closing of another pair of the diaphragms 3b. The aperture change lever 26d is used to input the directive of the operator relative to an aperture of the collimator 3a that limits the broadening of the X-ray that the X-ray tube 3 irradiates, and corresponds to the collimator operation input means of the present invention.

Whereby, the operator can adjust arbitrarily the shifting of the imaging system 3, 3a, 4, the rotation of the table 2, and the aperture of the collimator 3a.

Referring to FIG. 5, the operator uses the recording button 26b to input the directive relative to starting a video recording. Once the operator presses down the recording button 26b while the video imaging, the video recording in the memory element 28 as the video file from such timing starts. When the operator presses down the recording button 26b once more in such state, the video recording ends and the video file is completely created. The recording button 26b corresponds to the storing directive means of the present invention.

The operation panel 26 comprises the display element (monitor) 25 that displays the video. The operator imaging the video operates the operation panel 26 while watching the display element 25 with the eyes.

The operation panel 26 comprises a foot-switch (not shown in FIG.). Such foot-switch is a human interface that allows the operator to input the directive relative to starting the video imaging. Once the operator steps on the foot-switch, the video imaging starts. The video imaging is ongoing as long as the operator continues stepping on the foot-switch. Once the operator unlinks the foot from the foot-switch, the video imaging suspends.

(The Most Characteristic Structure of the Present Invention)

The inventor sets forth the most characteristic structure of the present invention. Specifically, the imaging system operation lever 26a and the recording button 26b of the present invention are unified. Further specifically, the recording button 26b is installed to the member of the imaging system operation lever 26a, which the operator grips, and furthermore specifically, the recording button 26b is installed to the superior portion (more accurately tip) of the member of the imaging system operation lever 26a, which the operator grips. The recording button 26b that is used to input the directive of the operator relative to the start of video recording is installed to the imaging system operation lever 26a. The recording button 26b is installed to the superior portion of the imaging system operation lever 26a. The recording button 26b can be installed to the side portion of the member, which the operator grips, of imaging system operation lever 26a.

In such way, the operator can press down the recording button 26b in the state while gripping the imaging system operation lever 26a. FIG. 6A is a schematic view illustrating the aspect in which the operator imaging the video is gripping the imaging system operation lever 26a. The pseudo-food that the subject M swallows passes the esophagus and reaches the stomach while imaging the video. Such video-imaging is carried out while tracking such movement. In addition, an incident in which the subject moves and the pseudo-food disappear from the sight likely takes place while imaging the video. In such incident, the imaging system 3, 3a, 4 must be shifted so that the pseudo-food appears in the center of the imaging sight while imaging the video. Accordingly, the operator imaging the video must continuously grip the imaging system operation lever 26a, and be ready to shift the imaging system 3, 3a, 4 any time.

FIG. 6B is a schematic view illustrating the state in which the operator presses down the recording button 26b. The recording button 26b is in-place at which the operator can press down even while gripping the imaging system operation lever 26a, so that the operator can press down the recording button 26b without unlinking the hand from the imaging system operation lever 26a.

In addition, as set forth referring to FIG. 5, the operation panel 26 comprises the table operation lever 26c and the aperture change lever 26d other than the imaging system operation lever 26a. With regard to the structure of the present invention, the inventor sets forth the reason why the system operation lever 26a rather than the levers 26c, 26d mounts the recording button 26b. The tilt of the table 2 and the aperture of the collimator 3a do not change while the fluoroscopic swallowing exam. Therefore, the operator does not always grip the levers 26c, 26d. On the other hand, the operator is always gripping the imaging system operation lever 26a due to necessity for shifting the imaging system 3, 3a, 4. When the imaging system operation lever 26a that the operator is always gripping mounts the recording button 26b, the operator can absolutely press down the recording button 26b by slightly moving the own hand (finger).

(Actual Video Imaging)

FIG. 7 is a flow-chart illustrating the method of the fluoroscopic swallowing exam using the X-ray imaging apparatus 1 according to the aspect of the present invention. When the fluoroscopic swallowing exam is performed using the X-ray imaging apparatus 1 according to the aspect of the present invention, the subject M first is loaded on the table 2 (the subject loading step S).

Once the operator steps on the foot-switch attached to the operation panel 26 and give the directive to start imaging the video, the X-ray tube control element 6 controls the X-ray tube 3 to irradiate an X-ray, and the FPD 4 starts detection of the X-ray that transmits through the subject M (the video-imaging starting step S2). The video denotes the fluoroscopic image of the subject M at the present time and is displayed on the display element 25. The display element 25 displays the present aspect of the fluoroscopic image of the subject M.

Since then the operator operates the imaging system operation lever 26a while watching the video that the display element 25 displays (the imaging system operation step S3). Along with such operation, the imaging system 3, 3a, 4 shifts relative to the table 2 and the fluoroscopic image of the subject M, which the display element 25 displays, moves as scrolling. According to such operation, the operator can continue to take video while tracking the pseudo-food that the subject M swallows.

Once the operator watching the video realizes the necessity of video recording, the operator presses down the recording button 26b attached to the imaging system operation lever 26a that the operator is gripping. The operator is, in advance, aware of that such recording button 26b is attached to the tip of the imaging system operation lever 26a, the operator can press down the recording button 26b without changing the line of the own sight from the display element 25. Once the operator presses down the recording button 26b, the memory element 28 stores the video as the video file (the recording starting step S4). Such video file is the video relative to the future direction denoting the change of the subject image from the timing when the operator presses down the recording button 26b.

Once the operator watching the video realizes the unnecessity of video recording, the operator presses down again the recording button 26b attached to the imaging system operation lever 26a that the operator is gripping. At this time, the operator can press down the recording button 26b without changing the line of own sight from the display element 25. When the operator presses down the recording button 26b, the video recording ends and the video file is completely created (the recording end step S5). In addition, instead of pressing down the recording button 26b, the operator steps off the foot-switch attached to the operation panel 26, so that the operator can end the video recording and create the complete video file.

As set forth above, with regard to the operation panel 26 of the present invention, the recording button 26b is installed to the imaging system operation lever 26a. The operator grips the imaging system operation lever 26a by a hand while the video imaging, so that the operator can operate the recording button 26b without unlinking the hand from the imaging system operation lever 26a. The operator is, in advance, aware of that the imaging system operation lever 26a mounts the recording button 26b, so that the operator can operate the recording button 26b while watching the display element 25 without changing the line of sight to the operation panel 26. Accordingly, with regard to the operation panel 26 according to the aspect of the present invention, when the operator imaging the video wants to start the video recording, the operator can start recording immediately without changing the line of the own sight, so that the scene needed for the diagnosis using the video can be absolutely recorded.

In addition, when the recording button 26b is installed to the superior portion of the imaging system operation lever 26a, it can be prevented that the operator erroneously operates the recording button 26b while operating the imaging system operation lever 26a.

The present invention is not limited to the above constitution and may work in the following alternative aspect.

(1) The inventor sets forth the fluoroscopic swallowing exam according to the aspect of the Embodiment, but the present invention can be applied to any general apparatus other than such apparatus for the exam.

(2) The present invention can be applied to the other apparatus having the different structure than the X-ray imaging apparatus set forth according to the aspect of the Embodiment.

(3) According to the aspect of the Embodiment, the video file that is stored is the video that starts at the timing when the recording button 26b is pressed down, but the present invention is not limited thereto. The video file having the end point that is the timing when the recording button 26b is pressed down can be recorded. Specifically, such video file of the alternative Embodiment is the video relative to the past direction denoting the change of the subject image until the timing when the recording button 26b is pressed down.

(4) According to the aspect of the Embodiment, the file that is recorded and stored is a video, a series of images, but the present invention is not limited thereto and can be applied to a plurality of sporadic images and even a single still image.

FIG. 8 is a timing chart illustrating a structure according to the aspect of the alternative Embodiment of the present invention. The X-ray imaging apparatus, according to the aspect of the present invention, comprises a volatile memory, and the video that is taken is temporarily stored as the video file having the starting point that is 30 seconds before the present time and the end point that is the present time. Such temporary video file is always renewed as long as the imaging is ongoing. Specifically, the past data from 30 seconds before until the present time stored in the volatile memory are overwritten by the updated data, so that the old data are deleted in series.

According to the present alternative Embodiment, when the operator presses down the recording button 26b, the 30 seconds video temporarily stored in the volatile memory is copied into the memory element 28 that is a non-volatile memory. At this time, such video file generated in the memory element 28 is the video denoting the change of the subject image from 30 seconds before the timing when the recording button 26b is pressed down (the press-down timing) until the press-down timing. Even such duration, the data in the volatile memory are renewed continuously.

Therefore, the temporary file on the volatile memory and the video file in the memory element 28 are the same file at the press-down timing. While time is running from the press-down timing, the renewal of the temporary file on the volatile memory proceeds, and after 30 seconds, all contents of the temporary file are renewed. On the other hand, the video file copied into the memory element 28 remains as-is, the operator can watch the video relative to the video file following the end of imaging.

### REFERENCE OF SIGNS

2 Table
3 X-ray tube (Radiation source)
3, 3a, 4 Imaging system
3a Collimator
25 Display element (Display means)
26 Operation panel (Console)
26a Imaging system operation lever
26b Recording button (Storing directive input means)
26c Table operation lever (Table operation input means)
26d Aperture change lever (Collimator operation input means)

Also, the inventors intend that only those claims which use the complete words “means for” are intended to be interpreted under 35 USC 112, sixth paragraph. Moreover, no limitations from the specification are intended to be read into any claims, unless those limitations are expressly included in the claims.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. A console, for a radiation imaging apparatus that comprises an imaging system that consists of a radiation source that irradiates a radiation and a detector that detects said radiation that transmits a subject, and a display means that displays fluoroscopic images of said subject as at least one of a real-time video and a still image, said console further comprising:
an imaging system operation lever that enables an actuation of an operator input directive relative to a shift of said imaging system along with said subject, and
a storing directive input means into which said operator inputs a directive to store a video, wherein said storing directive input means is installed on said imaging system operation lever.

2. The console, according to claim 1, wherein:
said storing directive input means is installed to a superior portion of said imaging system operation lever.

3. The console according to claim 2, further comprising:
a collimator operation input means into which said operator inputs a directive relative to an aperture of a collimator that limits a broadening of said radiation that said radiation source irradiates.

4. The console according to claim 3, further comprising:
a table operation input means into which said operator inputs a directive relative to a tilt of a table that said subject is loaded.

5. The console, according to claim 1, further comprising;
a collimator operation input means into which said operator inputs a directive relative to an aperture of a collimator that limits a broadening of said radiation that said radiation source irradiates.

6. The console, according to claim 1, further comprising:
a table operation input means into which said operator inputs a directive relative to a tilt of a table that said subject is loaded.

7. The console, according to claim 2, further comprising:
a table operation input means into which said operator inputs a directive relative to a tilt of a table that said subject is loaded.

8. A radiation imaging apparatus, comprising:
a console according to claim 1.

9. The radiation imaging apparatus, according to claim 5, wherein:
said radiation imaging apparatus stores at least one of a video and a still image from a timing when said operator inputs a directive through said storing directive input means.

10. The radiation imaging apparatus, according to claim 5, wherein:
said radiation imaging apparatus stores at least one of a video and a still image until a timing when said operator inputs a directive through said storing directive input means.
